(19) United States
(12) Patent Application Publication
(10) Pub. No.: US 2008/0214933 A1
(43) Pub. Date: Sep. 4, 2008

(54) Image-Based Planning Methods and Apparatus for Targeted Therapy

(75) Inventors: Heinrich Von Busch, Aachen (DE); Bernd Schweizer, Herzogenrath (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/064,372

(22) PCT Filed: Aug. 9, 2006
(86) PCT No.: PCT/IB06/52758

§ 371 Date: Feb. 21, 2008

Related U.S. Application Data
(60) Provisional application No. 60/711,299, filed on Aug. 25, 2005.

(51) Int. Cl. A61B 5/00

(52) U.S. Cl. 600/431; 382/128

(57) ABSTRACT

The present invention relates generally to biomedical devices. In particular, the present invention provides a method and apparatus for delivering a patient-specifically optimized treatment plan for targeted drug therapy that takes into account the individual 4-D biodistribution of the given targeted agent. An improvement in clinical outcome can be achieved in terms of disease response and survival rates and/or in terms of quality of life.

The present invention relates generally to biomedical devices and methods of providing more efficient drug treatment programs. In addition, the invention relates to a method and apparatus for 4-D imaging of radiolabeled drug biodistribution in a patient for an individualized drug treatment scheme and improved clinical outcomes.

Many human diseases, among them cancer, can be more specifically treated by targeted rather than non-targeted systemically administered drugs. However, targeted treatment schemes (e.g., immunotherapies) often involve strong patient-to-patient variability in the biodistribution of the targeted agent. As a consequence, the dosing requirement needed to achieve a maximum useful effect while limiting the side-effects may vary equally strongly, from patient to patient.

Nevertheless, in current practice dosing often is done on a one-dose-fits-all, or on a simple per-body-weight or per-body-surface basis, possibly with absolute minimum and/or maximum limits. This is in stark contrast to the planning effort routinely invested into the design of a patient-specific external radiation treatment scheme.

Present dosing methods for targeted therapy do not take into account the detailed spatial and temporal biodistribution of the targeted agent in the specific patient. This distribution may vary strongly from patient to patient, even if the drug is administered on a per-body-weight or per-body-surface basis. In order to stay on the safe side for even the most unfavorable biodistributions that may result from the interpatient variability, dose recommendations not related to the individual biodistribution are bound to be, on the average over different patients, lower than the patient-specific limit (as defined, e.g., by dose to risk organs). As a consequence, the effect of the drug will, on the average over different patients, fall short of what could be achieved by administering the drug to each patient at his or her specific dosage limits.

Even if patient-specific dosimetry is performed, it is at present most often based on conjugate-view planar imaging, even though, by principle, planar imaging cannot be expected to give quantitative results in the complicated 3-D geometry of the human body. One of the rare attempts to include individual tomographic information in dosimetry is described in Sgouros, G., et al., Patient-Specific, 3-Dimensional Dosimetry in Non-Hodgkin's Lymphoma Patients Treated with ¹³¹I-anti-B1 Antibody: Assessment of Tumor Dose-Response. J Nucl Med 44(2): 260-268, which is incorporated herein by reference in its entirety and for all purposes. The method by which individual 3-D information is collected by Sgouros et al. is SPECT imaging. However, the Sgouros et al. approach does not involve absolute 3-D dosimetry; rather, the 3-D activity distributions measured by SPECT are normalized on a per-tumour-region-of-interest basis to the cumulated counts per tumour as measured by planar imaging. Further, there is no real 4-D data basis, since 3-D data is typically taken only for one point in time.

Moreover, the image-based dosimetry in the Sgouros et al. approach is only conducted for tumor regions, but not for risk organs. Finally, from the dosimetry, no therapy planning is derived.

United States Patent Application Publication No. 2004/0225174A1 to Fuller et al. describes a method for optimizing the placement of a radiation dose using brachytherapy. The method requires ultrasound and CT imaging of the patient to determine correct surgical placement of radiation seeds. The invention suggests a method for planning the distribution of radiation seeds based on anatomical data only, and does not contemplate a method or apparatus for optimization of a therapeutic regimen based, in part, on patient-specific image modeling of target agent efficacy and pharmacologic data.

United States Patent Application Publication No. 2004/0023211A1 to Groen et al. describes a system for optimizing drug therapy using a combination of population based pharmacokinetic models, and phenotypic resistance testing. However, this method does not contemplate the use of 4-D imaging of targeted agent concentration in a patient, and expressly disavows the likelihood of performing quantitative monitoring of drug concentrations in each individual patient for each drug.

United States Patent Application Publication No. 2004/0015070A1 to Liang et al. describes a system for providing a 3-D simulation of an anatomical region, which allows a user to perform a “virtual” intervention to plan and assess potential risks and detrimental effects. However, the invention does not teach or suggest the imaging of targeted agent concentrations over time in a patient for the purpose of optimizing dose regimens.

United States Patent Application Publication No. 2002/0046010A1 to Wessol et al. describes a system for improved radiotherapy dosimetry planning through the modeling of the movement of a radioactive particle through a region in the patient. This method, however, does not teach or suggest steps that require modeling of patient-specific pharmacologic or pharmacokinetic data in determining the best overall treatment strategy.

United States Patent Application Publication No. 2002/0046010A1 to Wessol et al. describes a system and method for manufacturing implantable biomedical devices based on 3-D anatomical information but does not teach or suggest methods for modeling of patient-specific pharmacologic or pharmacokinetic data for optimizing efficacy of treatment for a given targeted agent.

U.S. Pat. No. 5,684,889 to Chen et al. describes a system and method for measuring the kinetics of an imaging agent by repetitively scanning the subject and generating an image of the intensity of the imaging agent, which can be used to develop a treatment plan for the subject. However, this method does not teach or suggest the use of a diagnostic imaging agent to generate 4-D biodistribution maps for predictive modeling of the biodistribution of a therapeutic agent. In addition, the '889 patent fails to contemplate the use of clinical and patient-specific data in order to optimize the patient-specific treatment regimen.

The descriptions of the features and shortcomings of the references cited above are intended for background purposes only, and are intended merely to highlight and give context to the advantages of the current invention.

The present invention relates to methods and an apparatus for converting 4-D medical images of a patient into a patient-specific treatment plan for targeted drug therapy that maximizes the useful effect while limiting side-effects. The nature of this treatment plan may range from a mere yes/no decision on the eligibility of one specific patient for one specific targeted drug treatment to a sophisticated treatment plan that uses a combination of therapies including targeted drug therapy.

One aspect of the invention relates to an apparatus for performing 4-D imaging. The apparatus comprises a 4-D image processing and registration unit for medical imaging data taken with a highly sensitive imaging modality including, for example, PET, SPECT, x-ray CT, MR, ultrasound, or optical imaging; a unit that estimates, possibly by means of pharmacokinetic models, from image intensities per voxel or 3-D region measured for a number of points in time the concentration curves over time for a diagnostic version of the targeted agent; a unit that estimates, from said concentration curves of the diagnostic version of the targeted agent, the temporal behavior and/or the integral over time of the concentration per voxel or 3-D region of the therapeutic version of the targeted agent; a unit that generates, on the basis of said estimated therapeutic agent concentration maps, a patient-specific therapeutic administration protocol; and an optional unit that calculates an estimate of the effect (and possibly toxicity) of the therapy plan.

Another aspect of the invention relates to methods for performing the 4-D biodistribution imaging. In one embodiment, the method of the invention relates to steps for performing imaging of the biodistribution of a drug or targeted agent in an individual over time to generate a patient-specific therapeutic plan. An aspect of this embodiment comprises administering a targeted agent to a patient in a diagnostic form, and taking a series of three-dimensional images over a period of time after the administration of the diagnostic targeted agent to the patient. High quality images may be obtained using, for example, PET, SPECT, x-ray CT, MR, ultrasound, or optical imaging according to standard imaging protocols. In another aspect, the present invention comprises imaging of the diagnostic targeted agent in a patient over a time course. One aspect of method includes estimating, such as through the use of a pharmacokinetic model, a first 4-D map of the concentration of the diagnostic form of the targeted agent. In another aspect of the invention, the method comprises using the first 4-D map of the concentration of the diagnostic form of the targeted agent, to estimate a second 4-D map of the concentration of the therapeutic form of the targeted agent, which also takes into consideration the differences in the physical and biokinetic properties between the diagnostic and the therapeutic forms of the agent. In a further aspect, the method of the invention comprises the step of using the estimated 4-D concentration maps for the therapeutic targeted agent to generate a patient-specific therapeutic administration protocol. In an additional aspect, the method of the invention optionally calculates an estimate of the effect and potential toxicity of the therapy plan.

The advantage of the present invention is that by a patent-specifically optimized treatment plan for targeted drug therapy that takes into account the individual 4-D biodistribution of the given targeted agent, an improvement in clinical outcome can be achieved in terms of disease response and survival rates and/or in terms of quality of life.

Additional advantageous features and functionalities associated with the systems, methods and processes of the present invention will be apparent from the detailed description which follows. The publications and other materials used herein to illuminate the background of the invention, and in particular cases, to provide additional details respecting the practice, are incorporated by reference, and for convenience are referenced in the appended bibliography.

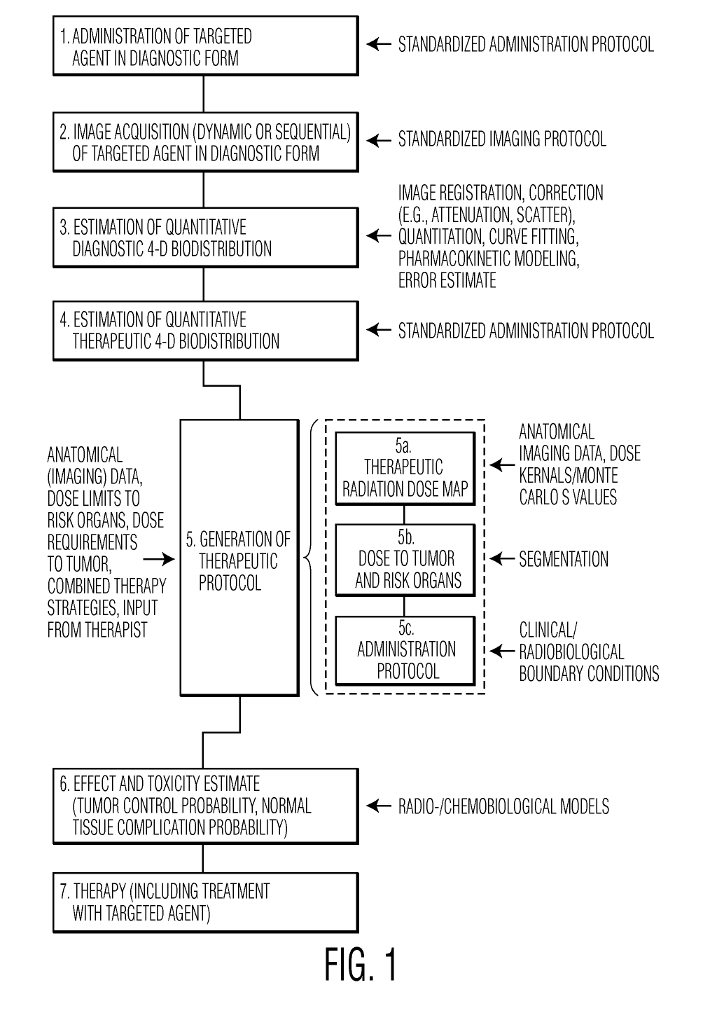

FIG. 1. Is a simplified flow diagram illustrating an overview of the present method and apparatus for 4-D image-based planning for targeted therapy.

Unless clearly indicated to the contrary, the following descriptions and definitions supplement definitions of terms known in the art.

Positron emission tomography (“PET” or “PET scan”), is a diagnostic examination that involves the acquisition of physiologic images based on the detection of radiation from the emission of positrons. Positrons are tiny particles emitted from a radioactive substance administered to the patient. The subsequent images of the human body developed with this technique are used to evaluate a variety of diseases.

PET scans are used most often to detect cancer and to examine the effects of cancer therapy by characterizing biochemical changes in the cancer. These scans can be performed on the whole body. PET scans of the heart can be used to determine blood flow to the heart muscle and help evaluate signs of coronary artery disease. PET scans of the heart can also be used to determine if areas of the heart that show decreased function are alive rather than scarred as a result of a prior heart attack, called a myocardial infarction. Combined with a myocardial perfusion study, PET scans allow differentiation of nonfunctioning heart muscle from heart muscle that would benefit from a procedure, such as angioplasty or coronary artery bypass surgery, which would reestablish adequate blood flow and improve heart function. PET scans of the brain are used to evaluate patients who have memory disorders of an undetermined cause, suspected or proven brain tumors or seizure disorders that are not responsive to medical therapy and are therefore candidates for surgery.

Before the examination begins, a radioactive substance is produced and attached, or tagged, to a biochemical compound, most commonly glucose, but sometimes water or ammonia. Once this substance is administered to the patient, the radioactivity localizes in the appropriate areas of the body and is detected by the PET scanner. The radioactivity often is very short-lived, and the amount is so small that it does not affect the normal processes of the body.

Different colors or degrees of brightness on a PET image represent different levels of tissue or organ function. For example, because healthy tissue uses glucose for energy, it accumulates some of the tagged glucose, which will show up on the PET images. However, cancerous tissue, which uses more glucose than normal tissue, will accumulate more of the substance and appear brighter than normal tissue on the PET images.

The radioactive substance is administered as an intravenous injection (although in some cases, it will be given through an existing intravenous line or inhaled as a gas). It will then take approximately 30 to 90 minutes for the substance to travel through the body and accumulate in the tissue under study. During this time, the patient must rest quietly and avoid significant movement or talking, which may alter the localization of the administered substance. After that time, scanning begins. This may take 30 to 45 minutes.

Because PET allows study of body function, it can help physicians detect alterations in biochemical processes that suggest disease before changes in anatomy are apparent with other imaging tests, such as CT or MRI. The value of a PET scan is enhanced when it is part of a larger diagnostic work-up. This often entails comparison of the PET scan with other imaging studies, such as CT or MRI.

Single photon emission (computed) tomography (“SPECT” or “SPET”) is a tomographic nuclear imaging technique producing cross-sectional images from gamma ray emitting radiopharmaceuticals (single photon emitters or positron emitters). SPECT data are acquired according to the original concept used in tomographic imaging: multiple views of the body part to be imaged are acquired by rotating the detector head(s) around a craniocaudal axis. Using suitable reconstruction techniques, cross-sectional images are then computed with the axial field of view FOV determined by the axial field of view of the gamma camera.

SPECT cameras are either standard gamma cameras which can rotate around the patient's axis or consist of two or even three camera heads to shorten acquisition time. A few systems use ring detector arrays similar to computed tomography CT but this type of system is not in widespread use. Data acquisition is over at least half a circle (180°) (used by some for heart imaging), but mostly over a full circle. Data reconstruction has to take into account the fact that the emitted rays are also attenuated within the patient; i.e., photons emanating from deep inside the patient are considerably attenuated by surrounding tissues.

Whereas in a CT scan absorption is the essence of the imaging process, in SPECT attenuation degrades the images. Thus, data of the head reconstructed without attenuation correction may show substantial artificial enhancement of the peripheral brain structures relative to the deep ones. The simplest way to deal with this problem is to filter the data before reconstruction. A more elegant but elaborate method used in triple head cameras is to introduce a gamma-ray line source between two camera heads, which are detected by the opposing camera head after being partly absorbed by the patient. This camera head then yields transmission data while the other two collect emission data. Note that the camera collecting transmission data has to be fitted with a converging collimator to admit the appropriate gamma rays.

Magnetic resonance imaging (“MR imaging”) is an imaging method using a strong magnetic field (B₀ field) and gradient fields to localize bursts of radiofrequency signals coming from a system of spins consisting of reorienting atomic nuclei (e.g., hydrogen nuclei, i.e. protons), after they have been disturbed by radiofrequency RF pulses. MR imaging produces high resolution, high contrast two-dimensional image slices of arbitrary orientation, but it is also a true volume imaging technique and three-dimensional volumes can be measured directly.

A “B₀ field”, also called magnetic flux density or induction, is the main magnetic field used in an MR imager. In current MR systems it has a constant value over time varying from 0.02 to 7 tesla. Field strengths of 0.5 T and above are generated with superconductive magnets. High field strengths have a better signal to noise ratio (SNR).

MR imaging is furthermore capable of quantifying velocity and higher order moments of motion (see flow quantification) and thus quantitating blood flow. Applications of MR imaging have steadily widened over the last decade. Currently it is the preferred cross-sectional imaging modality in most diseases of the brain and spine and has attained major importance in imaging diseases of the musculoskeletal system. MR imaging in the head and neck and pelvis has attained a substantial level of clinical use, and its applications in the abdomen, kidneys and chest are rapidly increasing with the advent of ultrafast MR imaging techniques.

MR imaging makes use of the NMR phenomenon, i.e. the fact that many nuclei exhibit a property called spin. These spins are orientated in an external magnetic field. External radiofrequency pulses disturb their orientated state and make them absorb energy, which is subsequently reradiated. The intensity of the reradiated signal is dependent on the radiating tissue and the pulse sequence used to disturb the spins. Since the NMR phenomenon has many contrast mechanisms, MR imaging is very rich in contrast. It is mainly determined by T₁ relaxation and T₂ relaxation processes, but other parameters such as the density of mobile protons (proton density), susceptibility effects, magnetization transfer (MT), diffusion and flow effects can also be made relevant contrast determining parameters. MR imaging requires spatial localization of the NMR-signal which is accomplished by using additional magnetic gradient fields. As a result, the signal behavior can be observed in small volume elements (voxels). Image data reconstruction is currently performed most frequently with the technique called Fourier transform imaging.

The signal measured in MR imaging is weak and can only be observed because of the very large number of proton spins in human tissue. The major concern in imaging is, therefore, to obtain an adequate signal to noise ratio SNR in the images. This can be accomplished in several ways. First, an increase in strength of the main magnetic field increases the SNR almost quadratically at lower field strengths, linearly at higher field strengths. Averaging multiple measurements also improves SNR but it only increases with the square root of time. A further widely used strategy to improve the SNR is the use of a local coil (surface coil).

NMR phenomenon (nuclear magnetic resonance) is a physical phenomenon understood within the framework of quantum theory based on the premise that many elementary particles such as the proton and electron carry an intrinsic angular momentum called spin. The NMR phenomenon deals with physical observations made with respect to the spins of nuclei, and of particular relevance in MR imaging of hydrogen H nuclei, i.e. protons.

The basic principles of NMR are as follows: the sample is placed in a strong external magnetic B₀ field with a field strength of 0.01 T-7 T in MR imaging. In this field, the nuclear spins in the sample orientate themselves either parallel with or antiparallel to the B₀ field. There is a slight energy difference between the two states given by γ [symbol] B₀, where γ is the gyromagnetic ratio and [symbol] is Planck's constant, divided by 2π. As a result there is a difference in the population of the parallel or spin up states relative to the antiparallel or spin down states, which is dictated by the distribution law of statistical mechanics in thermal equilibrium: the Boltzmann distribution. Since the energy difference between the two states is very small and of the order of 10⁻⁷ electron volt (eV), there is only a very small excess of the order of 1 in 10⁵ of spins in the spin-up than the spin-down state. Since organic samples (including the human body) contain an extremely large number of hydrogen spins per ccm, the result of thus placing such a sample in a strong magnetic field is nevertheless a perceptible macroscopic magnetization. When the sample is irradiated with an electromagnetic wave with a photon energy of that given by the energy between the two spin levels, the thermal equilibrium distribution will be disturbed: spins will be flipped and have the increased energy of the spin down level. An ensemble of many spins will therefore exhibit a net magnetization. This magnetization will behave like any classical magnetization. In fact, the flipping of the spins corresponds—in classical physical terms—to tilting the magnetization away from the direction of the main magnetic field (flip angle). The behavior of the spin ensemble is determined by the Bloch equations and leads to a precessional motion (precession) of the spins around the main magnetic field.

Two types of information can be obtained: in a classical NMR experiment, the frequency and thus the energy of the irradiating wave is swept across a certain range. Absorption or resonance (hence the name) occurs whenever a spin system is present, which has two (or more in some nuclei other than hydrogen) energy levels matching precisely the energy difference corresponding to the energy of the irradiating wave (electromagnetic wave). The corresponding frequency is called the La I/magnetization transfer. Magnetization, susceptibility, motion and flow effects and spin diffusion affect the signal intensity when the spin system is excited once or repetitively. These phenomena are of variable importance in MR imaging.

NMR spectroscopy of samples requires high field strengths of the main magnetic field as different resonance lines can be better separated from each other in an NMR spectrum. Medical MR imaging presently uses field strengths of up to 7 T.

Ultrasonic imaging is a term used to describe all diagnostic uses of ultrasound. All ultrasonography is based on the pulse echo method where an ultrasound transducer transmits brief pulses of ultrasound that propagate into the tissues. Each pulse travels in a narrow ultrasound beam, the shape of which is determined by the dimensions of the transducer, the ultrasound wavelength and the degree of mechanical or electronic focusing. The propagational speed (speed of sound) of the ultrasound pulses is determined by the elasticity and density of the medium, and is nearly constant in the soft tissues of the body (approximately 1,540 m/s). Whenever there is a change in acoustic impedance, some of the ultrasound is reflected or backscattered to the transducer as echoes. The duration of each pulse is in the order of 1-2 μs, and the pulse repetition frequency PRF is typically 1-5 kHz (1,000-5,000 pulses per second). Between pulse transmissions, i.e. approximately 99.7-99.9% of the time, the transducer serves as a detector of the echoes. The time interval (t) from pulse transmission to reception of an echo is used to determine the transducer-to-reflector distance or range (r): r=c t/2, where c is the speed of sound (1 540 m/s). The factor 2 is included to account for the round trip distance, 2r.

The detected echoes may be displayed in one-dimensional formats such as A mode or M mode, but in radiology, the two-dimensional B mode format is used almost exclusively. The transducer transmits the ultrasound beam, which is swept through the region of interest by mechanic or electronic means. In electronic array scanning the transmitted ultrasound beam is electronically steered. The echoes are detected by the piezoelectric crystal of the transducer, where mechanical deformation of the crystal is converted into radiofrequency (RF) electronic signals. The electronic signals go through several steps of signal processing and are then stored in the scan converter memory, where an image is built up and retained during the scan. The vertical location of the signals in the image memory are determined by the echo return times, and the horizontal locations by the position of the beam axis (scan line) when the echoes were detected. The output from the image memory is fed through a digital-to-analogue converter (DAC) and finally to a monitor where the B-mode image is displayed.

Ultrasound imaging thus comprises pure imaging techniques such as A mode, B mode and M mode, and various methods for imaging and/or measuring blood flow such as color Doppler sonography, power Doppler sonography, time domain correlation, continuous wave CW Doppler and pulsed Doppler ultrasound. Ultrasound uses sound waves with a frequency above the human adult hearing range, i.e. above 20,000 Hz. In medical diagnostics, ultrasound, mostly in the 2-15 MHz frequency range, is used to produce sectional images and to measure and image blood flow. In real-time ultrasound, ultrasound images are taken at frame rate high enough to follow physiological motion. A flicker-free display requires at least 16 frames per second. Real-time ultrasound images are produced by two basic types of instruments, 1) the mechanical scanner, and 2) the electronic array scanner. Compound real-time imaging is a digital ultrasound technique creating a compound image in real time. The image is generated by combining multiple steered scans with different scan line angulations thus combining the compound scanning of the old static B-scanner with modern real-time digital imaging.

Optical imaging is an important tool in life sciences for the detection of gene expression and protein-ligand interactions. Although many of the techniques are restricted to in vitro applications due to problems with optical access or labelling, optical imaging is being used increasingly for in vivo imaging as well. Absorption, reflectance, fluorescence, or bioluminescence can be used as the source of contrast. Currently, most of these techniques are primarily restricted to microscopic or surface imaging, or to experimental imaging in small animals, since the penetration depth of the light is limited. However, light within a small spectral window of the near infrared region (600-900 nm) can penetrate more than 10 cm into tissue, due to the relatively low absorption coefficient at these wavelengths. The lowest wavelength in this window is determined by the relatively high absorption of blood (haemoglobin), whereas absorption above 900 nm is due to water. Near-infrared (NIR) fluorescence and luminescence imaging make use of this optical window.

Usually, optical contrast agents have to be used to obtain high specificity and sensitivity. In fluorescence imaging a fluorescent probe (optical contrast agent) is activated by an external light source, and a signal is emitted at a different wavelength. The fluorescence signal can be captured with a high-sensitivity charge-coupled-device (CCD) camera. The sensitivity of fluorescence detection is very high, and in microscopic setups it is possible to detect single molecules. Although in vivo optical imaging is a powerful tool in cell culture studies or animal models, its current application to human health is generally limited to ‘close-to-surface’ structures, as in optical imaging of the eye and skin, or optical mammography.

For example, as disclosed in U.S. Pat. No. 5,602,397 to Pitts et al., an economical optical imaging device has been designed to provide a digitized radiative representation of ionization charges to a CCD camera. The combination of a radiation-sensitive device and a wavelength-shifting material enable the optical imaging device to transmit any absorbed combination of ultraviolet and infrared radiation to a particular pixel element, which the CCD camera converts to a visible light image. The light intensity from the imaging device results in images similar to those formed by x-ray film.

“Monte Carlo” modeling is a general technique for the investigation of real-world processes with random components by means of computer models. A typical example in medical imaging is the study of the changes in image characteristics such as linearity or resolution when changing the detector geometry. The principle of Monte Carlo modeling is that the behavior of all the components involved is mathematically described. In SPECT imaging, for example, an initial spatial distribution of tissue is required at a certain voxel resolution. For each voxel, the tracer activity and the attenuation coefficient is defined. From each voxel, gamma rays may be emitted at random time points according to the Poisson probability distribution function and sent into random directions. In each voxel which the gamma ray traverses there is a chance of interaction, i.e. the ray may be deflected and continue with less energy in a different direction. If a gamma ray succeeds in escaping the tissue it may enter the detector, or may miss it. Interaction in the detector also follows statistical laws which must be modeled, for example, the position where the gamma ray interacts, the number and direction of light photons it releases, and the way in which they are picked up by the phototubes. In the actual Monte Carlo simulation the computer program randomly (hence “Monte Carlo”) generates a radioactive decay in one of the voxels and calculates its fate when traveling through tissue towards the detector. The result of each decay is either a true event, a scattered event or a missed event. After a large number of such decays has been simulated, an image is reconstructed from the counts recorded in the “detector” and compared to the original object.

Internal dose calculations in nuclear medicine normally use the techniques, equations, and resources provided by the Medical Internal Radiation Dose (“MIRD”) Committee of the Society of Nuclear Medicine. The MIRD formalism uses a unique set of symbols and quantities to calculate the absorbed dose of radiation in any target organ per radioactive decay in any source organ. The calculations involve the energy emitted per radioactive decay, the fraction of the emitted energy that is absorbed in various target organs, the masses of these organs, and both the physical decay and biologic clearance of the injected radioactive material. Standardized mathematical models (phantoms) of the human body and standardized biokinetic models are also used. A computer program, MIRDose/OLINDA, calculates dose tables per unit administered activity of various radiopharmaceuticals. For a detailed review of MIRD concepts see, e.g., the AAPM/RSNA physics tutorial for residents: internal radiation dosimetry: principles and applications. Toohey, R. E., Stabin, M. D., Watson, E. E. Radiographics, 2000 March-April; 20(2):533-46; which is incorporated herein by reference in its entirety and for all purposes.

Pharmacokinetic modeling. Pharmacokinetic models are relatively simple mathematical schemes that represent complex physiologic spaces or processes. Accurate PK modeling is important for precise determination of elimination rate. The most commonly used pharmacokinetic models are: a) 1-compartment; and b) 2-compartment.

All drugs initially distribute into a central compartment (Vc) before distributing into the peripheral compartment (Vt). If a drug rapidly equilibrates with the tissue compartment, then, for practical purposes, we can use the much simpler one-compartment model which uses only one volume term, the apparent volume of distribution, Vd. Drugs which exhibit a slow equilibration with peripheral tissues, are best described with a two compartment model. During the initial, rapidly declining distribution phase, drug is moving from the central compartment to the tissue compartment. Elimination of drug is the predominant process during the second phase of the biphasic plot. Because elimination is a first-order process, the log plot of this phase is a straight line.

A one-compartment PK model may be used for drugs which rapidly equilibrate with the tissue compartment, e.g., aminoglycosides, however, a two-compartment model should be used for drugs which slowly equilibrate with the tissue compartment, e.g, vancomycin. A log scale plot of the serum level decay curve of a 1-compartment model yields a straight line, while a log scale plot of the serum level decay curve of a 2-compartment model yields a biphasic line. Failure to consider the distribution phase can lead to significant errors in estimates of elimination rate.

The present invention provides methods and an apparatus for performing four-dimensional (4-D) biodistribution imaging of a targeted agent to derive a patient-specific therapeutic treatment plan. As used herein, “targeted agent” is used generally to describe a pharmaceutical molecule, small molecule drug, radionuclide drug, protein, peptide, antibody, nucleotide, nucleic acid, and the like or any combination thereof, whether it be diagnostic or therapeutic, which has an interacting or binding target, for example, another molecule, a diseased, infected or injured organ, tissue or cell. The “diagnostic form” of a targeted agent may be, for example, a radiolabeled pharmaceutical, for example, a radionuclide, a contrast agent such as an x-ray contrast agent or other diagnostic material.

Radionuclides are used for diagnosis, treatment and research. Radioactive chemical tracers emitting gamma rays can provide diagnostic information about a person's anatomy and the functioning of specific organs. This is useful in performing in some forms of tomography, for example, SPECT, and PET scanning. Radionuclides are also a promising method of treatment in hemopoietic forms of tumors, while the success for treatment of solid tumors so far has been limited. In biochemistry and genetics, radionuclides are used to label molecules and allow tracing chemical and physiological processes occurring in living organisms, such as DNA replication or amino acid transport.

In a preferred embodiment, the apparatus of the invention comprises a first means for estimating 3-D concentration curves over time for a diagnostic targeted agent using pharmacokinetic models, from image intensities per voxel or three-dimensional (3-D) region measured for a number of points in time; a second means for estimating a 4-D concentration map for the therapeutic form of the targeted agent based on the first 4-D concentration map, and corrected for certain variables, for example, physical and biokinetic differences between the diagnostic and therapeutic forms of the targeted agent; and a third means for generating a patient-specific therapeutic administration protocol using data from the estimated 4-D concentration maps of the therapeutic targeted agent. In certain embodiments, the third means of the invention also includes consideration of other relevant data, for example, anatomical imaging, dose limits to risk organs, dose requirements to disease tissue, combined therapy strategies, input from a therapist, and individual patient characteristics such as body size, gender, age, physical and pathophysiologic states, genetics, environment, and concurrent therapies, and the like. In other embodiments, the third means utilizes a Monte Carlo-type calculation in order to achieve an accurate description of the dose profile.

Image intensities are determinable from a succession of images or measurements of image intensity taken over time. In any of the preferred embodiments, image intensity includes, for example, rate-of-change of image intensity, initial up-take image intensity, and cumulative image intensity. Image intensity for a given agent is a function of concentration, i.e., higher concentration results in greater intensity.

In still another embodiment, the apparatus of the invention optionally comprises a fourth means for predicting effect and toxicity of a therapeutic protocol using the therapeutic protocol data in conjunction with information regarding tumor control probability, drug efficacy, drug toxicity, and the like.

In any of the preferred embodiments of the apparatus of the invention, the means for performing the given function comprises a computer, computer system, computer software, an algorithm, or combination thereof, and optionally comprises a means for graphic display, for example, a computer monitor or a printer; and internet communication. The preferred embodiments optionally comprise at least one database which contains, for example, experimental concentration curve data, patient history data, drug risk or toxicity data, pharmacogenomic data, pharmacologic data, biokinetic data, drug efficacy data, drug interaction data, data pertaining a drug's physical characteristics, and the like.

In another preferred embodiment, the method of the invention includes steps for performing imaging of the 4-D biodistribution of a drug or targeted agent in an individual over time in order to generate a patient-specific treatment plan. One embodiment of this method comprises administering a targeted agent to a patient in a diagnostic form, and taking a series of three-dimensional images over a period of time after the administration of the diagnostic targeted agent to the patient. High quality images may be obtained using, for example, PET, SPECT, x-ray CT, MR, ultrasound, or optical imaging according to standard imaging protocols. Another embodiment of the method comprises imaging of the diagnostic targeted agent in a patient over a time course. In another embodiment the method includes estimating, such as through the use of a pharmacokinetic model, a first 4-D map of the concentration of the diagnostic form of the targeted agent. In still another embodiment, the method comprises using the first 4-D map of the concentration of the diagnostic form of the targeted agent, to estimate a second 4-D map of the concentration of the therapeutic form of the targeted agent, which also takes into consideration the differences in the physical and biokinetic properties between the diagnostic and the therapeutic forms of the agent. In a further embodiment, the method of the invention comprises the step of using the estimated 4-D concentration maps for the therapeutic targeted agent to generate a patient-specific therapeutic administration protocol. In an additional aspect, the method of the invention optionally calculates an estimate of the effect and potential toxicity of the therapy plan.

The invention further relates to systems, computer programs products, business methods, server side and client side systems and methods for generating, providing, and transmitting optimal dosage regimens for an individual patient.

The general descriptions and the following detailed descriptions are exemplary and are intended to provide further explanation of the invention and are not to be construed as limiting to the scope of the present invention.

### EXAMPLE #1

### Radioimmunotherapy of Cancer

The novel apparatus and method of the present invention can be utilized to improve patient outcomes. FIG. 1 is a simplified flow chart diagram illustrating an overview of the method of the present invention (steps 1-7). The breakdown of step 5 in sub-steps 5a-c relates to an exemplary embodiment for radioimmunotherapy of cancer.

As an initial step (with reference to FIG. 1) in the process of performing the method of the invention a diagnostic form of the targeted agent (e.g., radionuclide drug) is administered to a patient (1) according to a standardized administration protocol. The administration of the diagnostic form of the targeted agent is followed by acquisition of dynamic or sequential images of the distribution of the radionuclide drug in the patient according to a suitable imaging protocol (2), using, for example, PET or SPECT in conjunction with x-ray CT or MR for anatomical reference. Next, (3) the apparatus of the invention is used to estimate a quantitative 4-D concentration map over time using pharmacokinetic models, image intensities per voxel, and/or 3-D regions measured for a number of points in time. This step also includes correction of the 4-D concentration map for signal attenuation, scatter, limiting spatial or temporal resolution, the kinetics of the radionuclide, curve fitting, and kinetic modeling.

From the 4-D concentration maps generated using the radionuclide, 4-D biodistribution data (i.e., concentration per region over time) is estimated for the therapeutic form of the radiation immunotherapy dose (4), taking into account possible differences in the physical and biokinetic properties of the diagnostic and therapeutic form of the targeted agent.

In the present exemplary embodiment, a therapeutic radiation dose map (5a) per voxel or 3-D region is estimated from the 4-D concentration integral map for the therapeutic agent using, for example, Monte-Carlo methods, the S value approach according to the MIRD scheme, or kernel-based techniques. This step can be performed, for example, by an additional means adapted for calculating the therapeutic dose map from anatomical imaging data previously acquired according to the methods of the invention. Next, (5b) the doses of radiation absorbed by tumor and risk organs are calculated. This step can be performed, for example, by yet another means adapted for calculating the dose requirements to the tumor and absorbed radiation dose risk to organs. These data are then used by an additional means to generate a patient-specific, or patient-optimized therapeutic dosage administration plan (5c). This step optionally includes assessment of combined therapy strategies, and input from a clinical expert, for example, an oncologist.

Finally, all data are considered in view of therapeutic effect and toxicity estimates for each individual patient (6), for example, tumor control probability, normal tissue complication probability, genomic variations (e.g., small nucleotide polymorphisms) that could affect drug sensitivity, bioavailability, or metabolism. The resulting therapy protocol (7) will therefore be tailored on a patient-by-patient basis to deliver optimum clinical outcomes. Such an improvement will be obtained by delivering to a tumor, by means of a targeted radionuclide drug, the patient-specific maximum radiation dose that is permissible in view of the radiation dose limits to risk organs. It is also contemplated in alternative embodiments that diagnostic and therapeutic agent are one and the same substance, in which case, a low-dose administration of the therapeutic agent would be used for the diagnostic step.

It is understood that the embodiments, and detailed examples provided and described herein are for illustrative purposes only and that various modifications or changes in light thereof will be suggested to persons skilled in the art and are included within the spirit and purview of this application and are considered within the scope of the appended claims. All publications, patents, and patent applications cited herein are hereby incorporated by reference in their entirety for all purposes.

1. A 4-D medical imaging device comprising:
a first means for image processing to quantitatively correct and spatially map sequentially or dynamically taken 3-D image data onto an anatomic reference;
a second means for calculating an estimate from intensities per voxel or 3-D region, measured for a number of points in time, a corresponding concentration/activity curve over time for a diagnostic version of a targeted agent;
a third means for estimating, from said concentration curve of the diagnostic version of the target agent, the temporal behavior and/or the integral over time of the concentration per voxel or 3-D region of a therapeutic version of the targeted agent;
a fourth means for generating, on the basis of an estimated 4-D biodistribution of the therapeutic agent and/or its integral over time, a patient-specific therapeutic administration protocol; and an optional fifth means for calculating an estimate of the effect, toxicity or both of a particular treatment.

2. The device of claim 1, wherein said fourth means further comprises a means for calculating, from an estimated concentration integral map over time of the therapeutic agent, a therapeutic radiation dose map per voxel or 3-D region, possibly using calculation techniques including the S-value method according to the MIRD formalism, Monte-Carlo simulations, or kernel-based approaches.

3. The device of claim 2, further comprising a means that, on the basis of said therapeutic radiation dose maps, calculates the absorbed radiation doses to tumor and risk organs.

4. The device of claim 3, further comprising a means for generating a patient-specific therapeutic administration plan on the basis of the radiation doses to tumor and risk organs.

5. A method for performing medical imaging comprising the steps of:
administering a targeted agent in a diagnostic form to a patient in need thereof, according to a standardized protocol;
acquiring sequential or dynamic imaging data of the distribution of said diagnostic form of the targeted agent in the patient, according to a standardized protocol;
estimating a quantitative 4-D map of the concentration of the diagnostic form of the targeted agent;
estimating a quantitative 4-D map of the concentration of a therapeutic form of the targeted agent and/or its integral over time; and
generating, on the basis of said 4-D therapeutic concentration map and/or its integral over time, a patient-specific therapy protocol that includes the administration of the targeted agent.

6. The method of claim 5, wherein the step of estimating a quantitative 4-D map of the concentration of the diagnostic form of the targeted agent further comprises the step of correcting for statistical uncertainties comprising scatter, attenuation, limited spatial or temporal resolution, kinetics of the targeted agent, or any combination thereof.

7. The method of claim 5, wherein the step of estimating a quantitative 4-D map of the concentration of the therapeutic form of the targeted agent further comprises the step of taking into account possible differences in physical and/or biokinetic properties between the diagnostic and the therapeutic forms of the targeted agent.

8. The method of claim 5, wherein the step of generating a patient-specific therapy protocol further comprises the step of taking into account the estimated doses to target and risk organs and the uncertainty of these estimates, and further providing for interaction with a medical expert.

9. The method of claim 5, further comprising the step of calculating an estimate of the effect and/or toxicity of the patient-specific therapy protocol.
